(19) United States
(12) Patent Application Publication
(10) Pub. No.: US 2004/0137152 A1
(43) Pub. Date: Jul. 15, 2004

(54) Method and apparatus for producing a uniform film on a substrate

(76) Inventors: Donald Ward Parriott JR., Sparta, NJ (US); Sadath Ulla Khan, Randolph, NJ (US); Edward M. Janos, Basking Ridge, NJ (US)

Correspondence Address: John A. Artz, Artz & Artz, P.C., Suite 250, 28333 Telegraph Road, Southfield, MI, 48034, US

(21) Appl. No.: 10/339,450

(22) Filed: Jan. 9, 2003

(51) Int. Cl. A61L 2/00; B05D 3/12

(52) U.S. Cl. 427/351; 427/2.1; 427/355

(57) ABSTRACT

A coater and drier system for uniformly depositing a film on a substrate or backing member. A vacuum bar positioned under the substrate is utilized to smooth out and make the substrate uniform as it passes beneath a spreader bar member or roller member in order to provide a layer of film forming liquid material on a substrate of a prespecified thickness.

### TECHNICAL FIELD

The present invention relates to the production of a uniform film on a substrate and more particularly an apparatus and method for depositing a constant thickness film on a backing member.

### BACKGROUND OF THE INVENTION

Most drugs and medicines today are provided in the forms of pills or liquids. For example, prescription medicines are provided in pill form while cough medicines are either provided in liquid form or cough drop form. In addition, various medicines for indigestion, such as antacids, are also provided in tablet or liquid form.

With most of these medicines, it is necessary to have the bottle, container, or a liquid solution of some sort, such as water, in order to take or dispense the medicine. This often makes it inconvenient for travel purposes or if the medicine has to, or needs to be, taken while the person is not at home or near the medicines. There, thus, is a need for another form in which to provide and/or dispense drugs and medicines which is easier to transport and take, and in which a liquid used to assist in swallowing, such as water, is not needed.

### SUMMARY OF THE INVENTION

It is an object of the present invention to provide an apparatus and method for providing drugs, medicines, and other materials in a form which is easier to transport and take.

It is another object of the present invention to provide a drug, medicine, or the like in a sheet-type form which can be packaged and dispensed in a quick and easy manner.

It is still another object of the present invention to provide drugs, medicine, and the like in a uniform film-type material with a consistent thickness and thus a consistent dosage.

These and other objects, as well as other advantages and benefits, are provided by the present invention. In accordance with the present invention, drugs, medicines, and other materials, are provided in a film-type form which can be easily carried, transported, and taken when it is necessary to do so. The materials can be, for example, a single or multiple drug substance, a medicine, a breath freshener, an antacid, an analgesic, an antihistamine, etc. The material for the film includes a film forming polymer, together with the particular drug or medicine involved, both dispensed in a solvent, such as water, alcohol, acetone, or the like. The liquid material is spread uniformly on a backing substrate, such as a paper material. A spreader bar or roller dispenses the liquid material evenly on the backing member.

The backing member or substrate is held in a planar configuration by a vacuum bar member on which the backing substrate is positioned when the liquid film material is applied to it. A frame mechanism holds the backing member in a stretched and tight position. It is important to provide a uniform thickness of film on the substrate material particularly where drugs or medicines are included in the material so that proper and consistent dosages are provided from area to area on the sheet of material when it is formed.

The backing member with the liquid composition on it are positioned in a drying member, such as a housing or chamber and air dried until a solid film is formed on the backing member. Thereafter, the film material is separated from the backing member, cut into individual dosage portions, and packaged in a portable container or the like for subsequent use. Thereafter, when it is appropriate to take the drug, medicine, etc., one of the pieces of film material can be dissolved in the person's mouth. Another liquid is not needed in order to wash down the drug or medicine, and it is not necessary for the person to carry with him a container with an assortment of pills or a bottle of liquid.

### BRIEF DESCRIPTION OF THE DRAWINGS

FIG. 1 is a perspective view illustrating a liquid coating and drying mechanism in accordance with the present invention;

FIG. 2 is a perspective view of a vacuum bar member in accordance with the present invention;

FIG. 3 is a cross-sectional view of the vacuum bar member as shown in FIG. 2, with the cross-section being taken along line 3-3 and in the direction of the arrows; and

FIG. 4 is an exploded view of the vacuum bar member in accordance with the present invention.

### DESCRIPTION OF THE PREFERRED EMBODIMENT(S)

The present invention is directed toward the formation of a consistent and uniform film member on a substrate so that each portion or area of the film has the same amount or dosage of a drug, medicine or the like. For this purpose, the substrate or backing member should be as smooth and planar as possible and the coating system or mechanism used to apply the liquid material from which the film is formed also needs to apply a uniform amount of material on the substrate. If the backing member is not flat or straight, then the thickness of the film material, and thus the amount of drug or medicine in the various areas of the sheet will not be consistent.

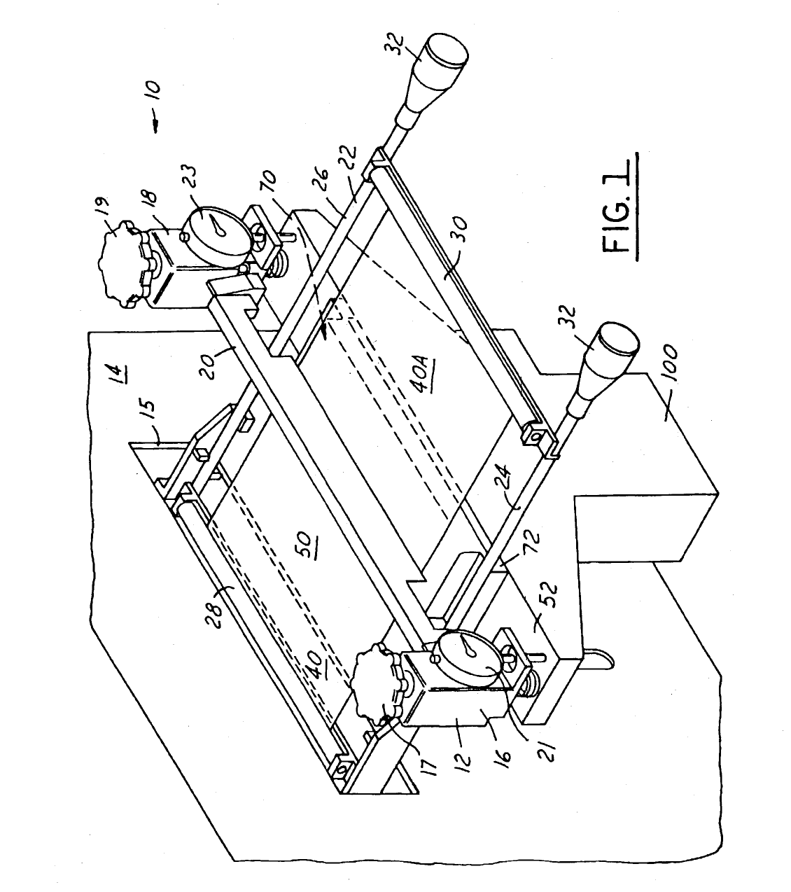

A coating and drying mechanism and system for use in the present invention is shown in FIG. 1 and designated generally by the reference numeral 10. The system 10 includes a coating mechanism 12 and a drying mechanism 14. The drying mechanism 14 can be any type of heating chamber or housing and preferably has a source of heated air in order to heat and dry materials which are placed inside of it. Heated air from an air source (not shown) is introduced into the housing of the drier 14 and used to dry the liquid film forming material.

The liquid film coating mechanism 12 includes a pair of spreader bar adjustment members 16 and 18 positioned on opposite sides of a spreader member 20. A frame member 22 is utilized to position the substrate and film forming material in the coating mechanism 12 and drier mechanism 14. The frame 22 includes a pair of side rod or rail members 24 and 26 and a pair of end holder members 28 and 30. The frame member 22 is manually movable and positionable by use of handles 32.

A substrate or backing member 40 is positioned in the frame member 22 and stretched and held tightly in place by the four members which make up the frame, namely the side rods or rails 24, 26 and the end holder members 28 and 30. The backing member can be made of any conventional material which is suitable for the process, such as a paper material. Also, preferably the backing member has a thickness on the order of 0.5 mm-1.0 mm and preferably 0.5 mm.

A liquid film forming material 50 is poured or sprayed onto a first section 40A of the backing member. The sprayer member 20 is positioned a preset distance above the plate member 52 in order to form a liquid layer on the backing member of a predetermined thickness. In this regard, as the frame member 22 is forced or slid between the plate member 52 and spreader member 20 into the opening 15 in the drier mechanism 14, the spreader member 20 spreads out the film forming liquid material 50 on the backing member 40, thus providing a smooth and uniform thickness coating of material on the substrate as it enters the drying member 14. The frame member can be manually inserted between the plate and spreader members and into the drier mechanism, or it can be automatically or mechanically inserted by an electronic or mechanical system (not shown).

The film forming material 50 is applied to the backing member in a liquid condition. The material includes a film forming polymer, such as Pollulan, a solvent ingredient, such as water, alcohol, or acetone, and a predetermined amount of a single or multiple drug substance, medicine or the like. Preferably, the liquid film forming material has a 15-40 percent solid content and a viscosity of 3,000-10,000 cps.

The liquid film forming material can be positioned or sprayed on the backing member 40 in the frame member 22 in any conventional manner. In addition, a roller member can be utilized in place of—or in combination with—the spreader member 20 in order to spread out the material to a consistent thickness on the backing member.

Once the frame member, backing member, and liquid film forming material are passed under the spreader member 20 and into the drier mechanism 14, the liquid material is dried and converted to a solid material. In this regard, the solvent ingredient in the film forming material is removed by evaporation which causes the remaining ingredients to dry out and solidify. Typically, the film forming material in a liquid condition has a thickness on the backing member of about 0.1-0.8 millimeters (mm). Once the liquid material has dried, the resulting solid film material has a thickness on the order of 0.3 mm-0.15 mm.

The temperature in the drier member 14 is preferably on the order of 40-120° C. and preferably in the range of 55-65° C.

It is also possible to apply the liquid film forming material to the substrate by first spraying or coating the material on a roller member or apparatus and then transferring the liquid from the roller member to the backing member. This can often provide a more uniform layer of material on the substrate.

A coater and drier mechanism which can be used with the present invention is a Mathis “Labcoater.” The Mathis product is made by Warner-Mathis AG, Zurich, Switzerland.

Hand wheels 17 and 19 on the spreader bar adjustment members 16 and 18 are utilized to raise and lower the spreader bar 20 and thus provide the desired thickness of film on the substrate. A mechanical or automated system could also be utilized for this purpose. Measuring gauges 21 and 23 can be used to aid in determining proper placement of the spreader member 20.

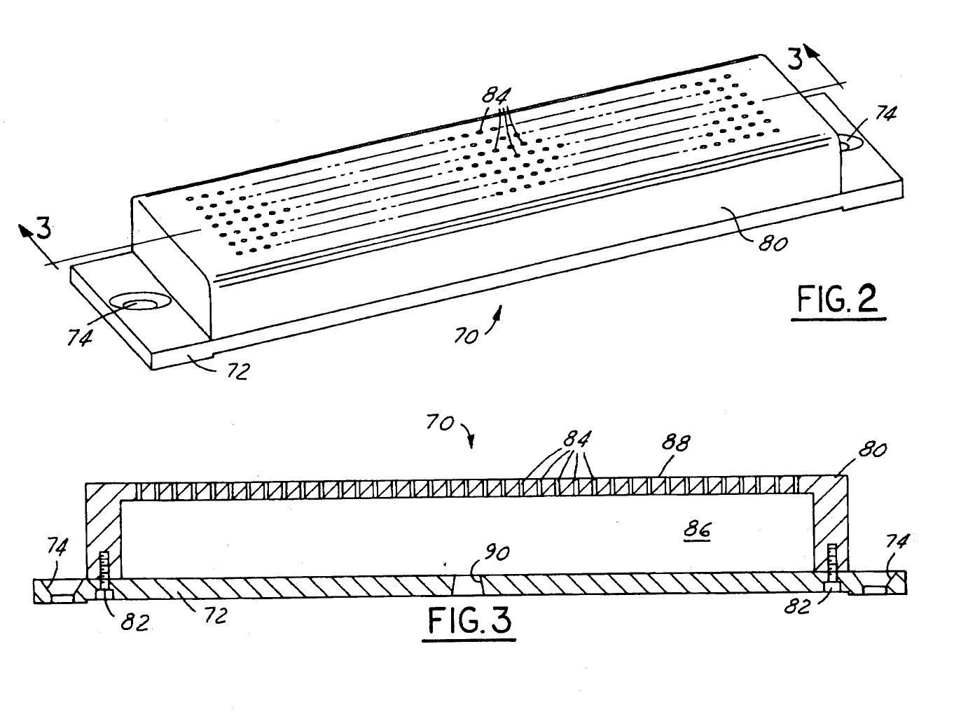

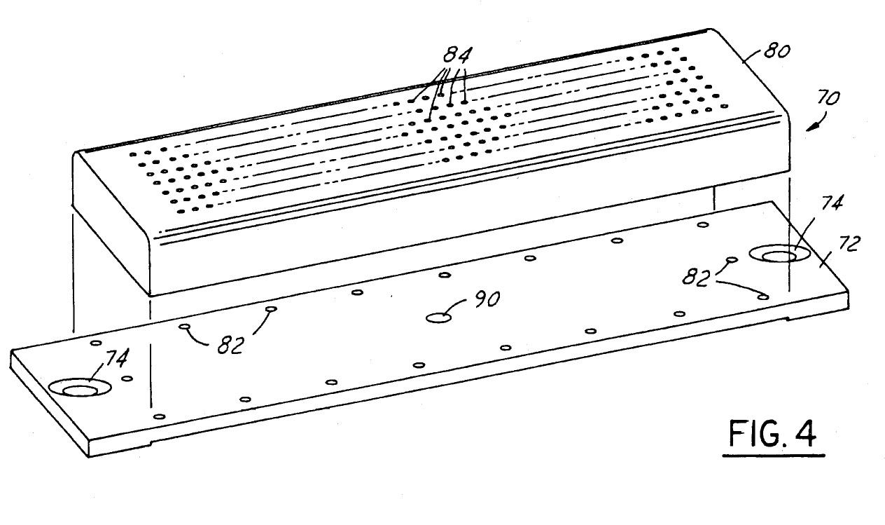

A vacuum bar member 70 is used to smooth out and hold down the backing member 40 as it passes under the spreader bar member 20. The vacuum bar member 70 is shown in detail in FIGS. 2-4. The vacuum bar member 70 includes a base plate 72 which is secured to the plate member 52. The base member 72 can be secured to the plate member 52 by bolts or other fasteners positioned through openings 74 in the base member 72.

A top or cap member 80 is secured to the base member 72. The top member 80 is secured to the base member by a plurality of fasteners which are positioned in aligned openings 82 through the plate member and walls of the top member (see FIG. 3). The top member 80 had a plurality of small holes or openings 84 in its top or upper surface. A cavity 86 is also formed in the top member when it is attached to the base member 72.

When the vacuum bar member 70 is positioned on the plate member 52, a vacuum is applied or created which holds the backing member tightly against the upper surface 88 of the top member 80. The vacuum is pulled through opening 90 in the base member 72. The plate member 52 is positioned on a manifold 100 through which the vacuum is pulled or formed. An opening in the plate member 52 aligns with the opening 90 in the vacuum bar member 70 and is utilized to draw the vacuum.

The present invention can be utilized with any process wherein it is desired to have a material spread as uniformly as possible on a substrate. The material can be a liquid, semi-liquid or solid. The principal ingredient in the material can be any ingredient in which consumers may want to consume, such as a drug or medicine, a breath freshener, an antacid, an analgesic, an antihistamine, a cough suppressant, or the like. A single or multiple drug substance or other ingredient can also be utilized.

While particular embodiments of the invention have been shown and described, numerous variations and alternate embodiments will occur to those skilled in the art. Accordingly, it is intended that the invention be limited only in terms of the appended claims.

What is claimed is:

1. A method of forming a film member having uniform thickness;
providing a backing member;
applying film material in a liquid form on said backing member;
spreading said liquid film material evenly on said backing member;
applying a vacuum suction to said backing member in order to provide a planar surface for said liquid film material; and
drying and solidifying said liquid film material into a solid film member.

2. The method as recited in claim 1 wherein said backing member is made of a paper material.

3. The method as recited in claim 1 wherein said liquid film material includes an aqueous solvent.

4. The method as described in claim 1 wherein said liquid film material includes a material selected from the group comprising a drug, a medicine, a cough suppressant, a breath freshener, an analgesic, an antihistamine, and an antacid.

5. The method as described in claim 1 wherein said liquid film material is spread by a spreader bar member.

6. The method as recited in claim 1 wherein said vacuum suction is applied by a vacuum bar member.

7. The method as recited in claim 1 wherein said drying and solidifying step is performed in a housing and wherein heated air is introduced into said housing.

8. The method as recited in claim 1 further comprising the step of removing said backing member from said solid film member.

9. The method as set forth in claim 1 further comprising the step of dividing said solid film member into smaller separate pieces.

10. The method as set forth in claim 1 wherein said liquid film material includes a drug ingredient and wherein said solid film member is removed from said backing member and divided into smaller separate single dosage-sized pieces.
